(19) United States
(12) Patent Application Publication
(10) Pub. No.: US 2007/0189919 A1
(43) Pub. Date: Aug. 16, 2007

(54) Method for cleaning a nebulizer

(75) Inventors: Ivan R. Prince, Chichester (GB); Richard Potter, Chichester (GB)

Correspondence Address: MICHAEL W. HAAS;RESPIRONICS, INC., 1010 MURRY RIDGE LANE, MURRYSVILLE, PA, 15668, US

(73) Assignee: Respironics Respiratory Drug Delivery (UK) Ltd., Bognor Regis (GB)

(21) Appl. No.: 11/699,300

(22) Filed: Jan. 29, 2007

Related U.S. Application Data
(60) Provisional application No. 60/763,616, filed on Jan. 31, 2006.

(51) Int. Cl. A61L 2/18; B01D 11/04

(52) U.S. Cl. 422/28; 422/255

(57) ABSTRACT

A method for cleaning a medicament from a portion of a nebulizer comprising soaking the portion of the nebulizer for a predetermined time in a cleaning solution having an enzyme dissolved therein, washing the cleaning solution from the portion of the nebulizer, and rinsing the portion of the nebulizer after the washing.

### CROSS-REFERENCE TO RELATED APPLICATIONS

This application claims priority under 35 U.S.C. § 119(e) from provisional U.S. patent application No. 60/763,616 filed Jan. 31, 2006 the contents of which are incorporated herein by reference.

### BACKGROUND OF THE INVENTION

1. Field of the Invention

The present invention pertains to devices used to deliver a medicament to a patient in aerosol form, and in particular, to a method of cleaning devices used to deliver medicaments.

2. Description of the Related Art

There are three basic categories of devices for delivering a medicament in aerosol form directly to the lungs of a patient during inhalation: metered-dose inhalers (MDI); dry powder inhalers (DPI); and nebulizers. Generally, medicaments available for use in a MDI are provided in a pressurized formulation (e.g., a hydrofluoroalkane formulation; a chlorofluorocarbon formulation), medicaments available for use in a DPI are provided in a dry powder formulation, and medicaments available for use in a nebulizer are provided in an aqueous solution formulation.

Due to their ease of use, many patients prefer nebulizers for the delivery of aerosolized medicaments. Nebulizers may employ different mechanisms for aerosolizing a medicament. For example, pneumatic (or jet) nebulizers employ compressed air to disperse the liquid into aerosol form. An ultrasonic nebulizer, in contrast, employs an electrical source to excite a piezoelectric element which generates high frequency vibrations. These vibrations cause small droplets to separate from the surface of the medicament, thus forming an aerosol. Vibrating mesh technology (VMT) nebulizers employ a mesh having a number of microscopic apertures therein. A vibration is induced in the mesh (or in another component within the nebulizer) which causes the aqueous-based medicament to be extruded through the mesh apertures. The aqueous-based medicament is ejected from the mesh apertures in aerosol form.

VMT nebulizers have gained in popularity among patients. VMT nebulizers, however, possess some drawbacks. For example, some medicaments delivered by a VMT nebulizer tend to accumulate on the surface of the nebulizer mesh. This accumulation may occlude the microscopic apertures in the mesh, thereby reducing the effectiveness of the nebulizer. For example, total blockage of one or more apertures may decrease the efficiency of the device or ultimately prevent any medicament from being dispensed to the patient. In addition, partial blockage of one or more apertures may negatively affect the particle size or time of delivery, thus possibly reducing or eliminating the beneficial effects of the prescribed therapy.

Alpha-1 Antitrypsin (A1AT) is one example of a medicament that tends to accumulate on the mesh. A1AT is a protein that protects the lungs from damage caused by the presence of excessive amounts of neutrophil elastase. Because A1AT has a tendency to accumulate, the mesh must be cleaned regularly to prevent the A1AT from occluding the mesh apertures.

A typical nebulizer cleaning protocol requires that the nebulizer be disassembled and the mesh removed. The mesh is then washed with warm soapy water. Afterwards, the mesh is rinsed in tap water and allowed to air dry. After the mesh has dried, it is re-inserted into the nebulizer which thereafter is reassembled for use. This cleaning protocol, however, has been found to be ineffective. Even when this cleaning protocol is implemented after each A1AT treatment, the mesh apertures still become occluded.

Other cleaning protocols have also been suggested for cleaning the mesh. One such cleaning protocol, for example, requires a water/vinegar solution to be added to the medicament chamber of the nebulizer and the nebulizer operated for approximately 45-60 seconds. The nebulizer is then disassembled and the mesh removed. Next, the mesh is soaked in the vinegar/water solution for approximately 10 to 15 minutes. After soaking, the vinegar/water solution is rinsed from the mesh with warm tap water and the mesh is allowed to air dry. The mesh is then re-inserted into the nebulizer which thereafter is reassembled for use.

Another cleaning protocol suggests that a water/vinegar solution be added to the medicament chamber of the nebulizer and operated for approximately 45-60 seconds. The nebulizer is then disassembled and the mesh is removed. Next, the mesh is submerged in a bowl of distilled water which is placed in a microwave oven and heated on “high” for approximately 3 minutes. The mesh is left to soak in the heated distilled water for approximately 30 minutes. After soaking, the mesh is allowed to air dry. The mesh is then re-inserted into the nebulizer which thereafter is reassembled for use.

Still other cleaning protocols suggest that the mesh be placed in a high-temperature dishwasher, in an autoclave, and/or in an ultrasonic cleaner. None of the above-mentioned cleaning protocols, however, even if performed after each treatment, adequately prevent medicament from accumulating on the mesh and/or adequately remove medicament that previously accumulated on the mesh. The mesh of a nebulizer used to aerosolize a drug such as A1AT may become occluded, for example, after approximately 10 to 20 treatments, even if the nebulizer is thoroughly cleaned using any one of the protocols discussed above. As a result, a user must dispose of the occluded mesh and purchase/install a replacement mesh.

Accordingly, a need exists for a method of cleaning a nebulizer that improves upon existing cleaning protocols. More specifically, a need exists for a method of cleaning a VMT nebulizer that prevents and/or removes the accumulation of medicaments on, and extends the useful life of, the nebulizer mesh.

### SUMMARY OF THE INVENTION

Accordingly, it is an object of the present invention to provide an improved method for cleaning a nebulizer that overcomes the shortcomings of conventional methods of cleaning. This object is achieved according to one aspect of the present invention by providing a method for cleaning a medicament from at least a portion of a nebulizer comprising soaking the portion of the nebulizer for a predetermined time in a cleaning solution having an enzyme dissolved therein, washing the cleaning solution from the portion of the nebulizer, and rinsing the portion of the nebulizer after the washing.

Another aspect of the invention relates to a method for removing a protein from a nebulizer mesh comprising soaking the mesh in a cleaning solution for a predetermined time, wherein the cleaning solution has a proteolytic enzyme dissolved therein, washing the cleaning solution from the mesh, and rinsing the mesh after the washing.

These features and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and not intended as a definition of the limits of the invention.

### BRIEF DESCRIPTION OF THE DRAWINGS

FIG. 1 is an isometric view of a vibrating mesh technology nebulizer;

FIG. 2 is an exploded view of the nebulizer of FIG. 1;

FIG. 3 is an isometric view of a mesh for the nebulizer shown in FIG. 1;

FIG. 4 is a flowchart illustrating an operational process for cleaning a nebulizer according to an exemplary embodiment of the present invention;

FIG. 5 is an image of the top surface of a mesh prior to exposure to an A1AT treatment and/or a cleaning protocol;

FIGS. 6-8 are images of the top surface of a mesh after exposure to a single A1AT treatment;

FIGS. 9-11 are images of the top surface of a mesh after exposure to ten A1AT treatments;

FIGS. 12-13 are images of the bottom surface of the mesh of FIGS. 9 -11;

FIGS. 14-15 are images of the top surface of a mesh after exposure to ten A1AT treatments and cleaning by a known cleaning protocol;

FIG. 16 is an image of the top surface of a mesh after being exposed to ten treatments of A1AT and cleaning by a cleaning protocol according to an exemplary embodiment of the present invention; and

FIGS. 17-19 are images of the top surface of a mesh after being exposed to fifteen double run treatments of A1AT and cleaning by a cleaning protocol according to an exemplary embodiment of the present invention.

### DETAILED DESCRIPTION OF THE EXEMPLARY EMBODIMENTS

Directional phrases used herein, such as, for example, horizontal, vertical, left, right, clockwise, counterclockwise, top, bottom, up, down, and derivatives thereof, relate to the orientation of the elements shown in the accompanying drawings and are not limiting upon the claims unless expressly recited therein.

As employed herein, the term “number” shall mean one or more than one, and the singular form of “a”, “an”, and “the” include plural referents unless the context clearly indicates otherwise. Additionally as employed herein, the statement that two or more parts are “connected” or “coupled” together shall mean that the parts are joined together either directly or joined together through one or more intermediate parts, whereas the statement that two or more parts are “attached” shall mean that the parts are joined together directly.

The present invention is generally directed to a method of cleaning a nebulizer and, more particularly, directed to a method of cleaning a vibration mesh technology (VMT) nebulizer. The method of cleaning prevents substances, such as protein based medicaments, from accumulating within the nebulizer and removes substances, such as protein based medicaments, that have accumulated within the nebulizer. For example, the method removes the accumulation of a medicament on the nebulizer mesh thereby promoting the free flow of the medicament through the mesh and extending the useful life of the mesh. Although discussed in conjunction with VMT nebulizers, it is contemplated that the present invention can be employed with other types of nebulizers (e.g., pneumatic nebulizers; ultrasonic nebulizers; etc.).

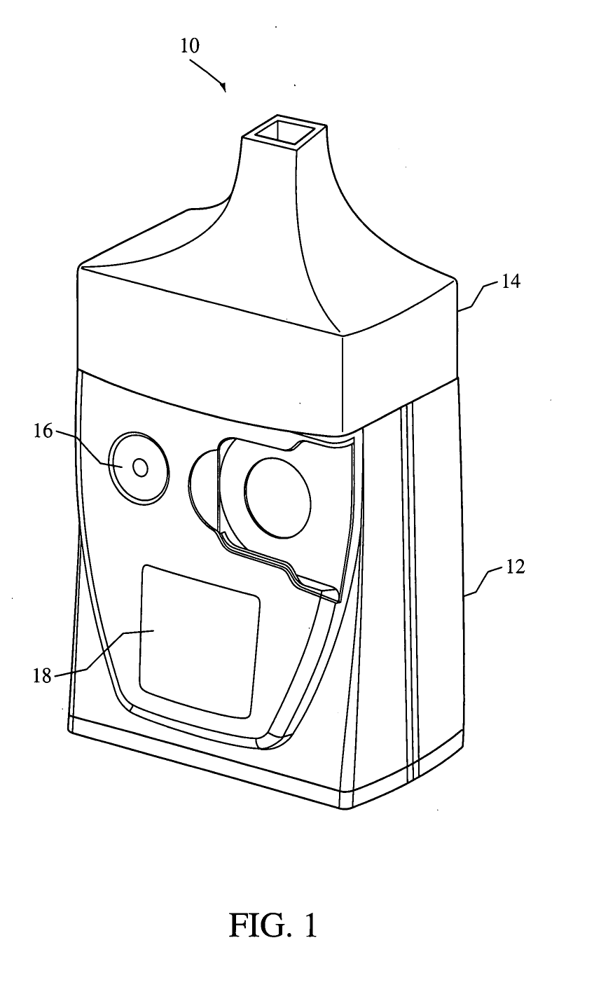

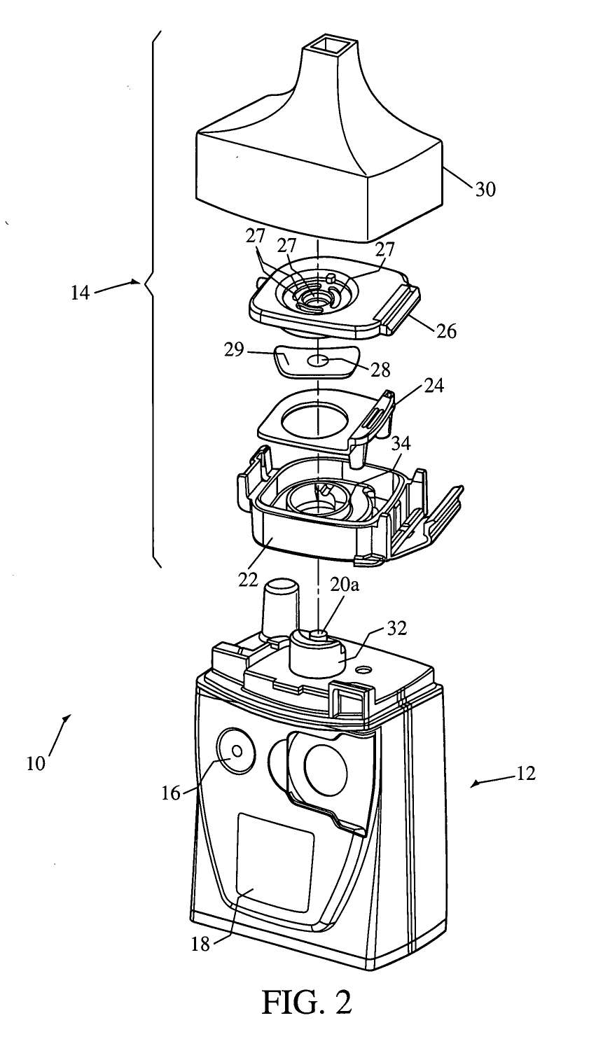

FIGS. 1 and 2 are isometric and exploded views of a VMT nebulizer 10. One example of such a nebulizer 10 is an I-neb® device distributed by Respironics, Inc. of Pittsburgh, Pa. The nebulizer 10 includes a base portion 12 and an upper portion 14. Base portion 12 includes a power supply source (not shown) such as a rechargeable battery pack, an on/off switch 16, a display 18, and a horn device 20. The base portion 12 houses circuitry (not shown) used to control the operation of the nebulizer 10. In the exemplary embodiment, the circuitry includes a microprocessor which is structured to access, without limitation, routines, instructions, and control data stored, for example, on a disc and/or a memory. The instructions may include, for example and without limitation, the dosage amount, the dosing frequency, and the number of doses that may be delivered; whereas the control data may include, for example and without limitation, the medicament lot number and expiration date. The microprocessor is adapted to execute the routines in response to the instructions and control data and, in conjunction with the other circuitry, cause the nebulizer 10 to dispense the proper dose of medicament to a patient.

The upper portion 14, which is removably connected to the base portion 12, includes a medicament chamber 22, a guide 24, a flexible housing 29, a chamber lid 26, and a mouthpiece 30. The horn device 20 extends from boss 32 which fits into opening 34 of chamber 22. Chamber 22 is structured to hold an aqueous-based medicament therein. The chamber 22 along with guide 24 directs the medicament towards horn device 20. Flexible housing 29 holds a mesh 28 having an upper surface 28a and a lower surface 28b.

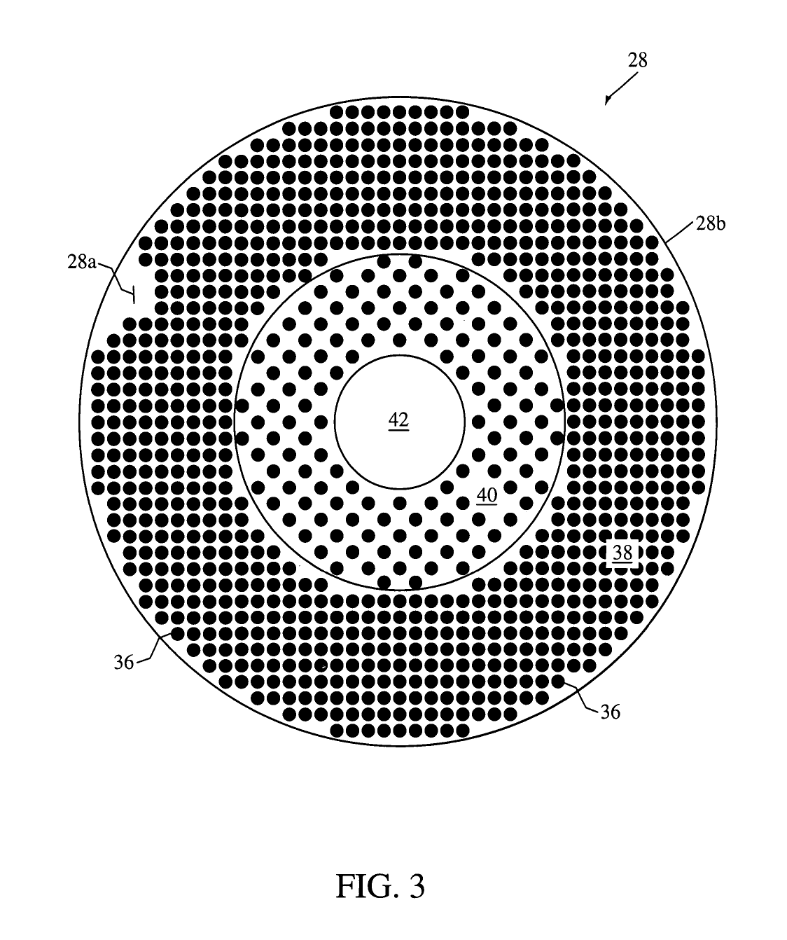

The horn device 20 includes a piezoelectric element (not shown) that is in electrical communication with the circuitry (not shown) contained within the base portion 12. The horn 20 and piezoelectric element of the base portion 12 vibrates the mesh 28 of to form a nebulization mechanism. More specifically, the lower surface 28b (FIG. 3) of the mesh 28 contacts a top surface 20a of the horn 20. The piezoelectric element is structured to oscillate when driven by the circuitry contained within base 12. A vibratory motion caused by the oscillation of the piezoelectric element is transferred to the horn 20 and to the mesh 28. The vibratory motion causes the aqueous-based medicament within chamber 22 to pass through a number of apertures 36 (FIG. 3) of mesh 28. The medicament leaving the apertures 36 at the upper surface 28a (FIG. 3) of the mesh 28 is aerosolized. Upon exiting the apertures 36, the aerosolized medicament passes from the upper surface 28a of mesh 28 through openings 27 and into mouthpiece 30. Once within the mouthpiece 30, the aerosolized medicament can be inhaled by a patient.

It should be noted that the upper portion 14 of the nebulizer 10 can be disassembled into its component parts, for example, during cleaning. Particularly, the chamber lid 26 and mesh 28 may be separated from the other components of the nebulizer 10. The mesh 28 may remain housed within the chamber lid 26 during cleaning and/or the mesh 28 may be removed from the chamber lid 26 and cleaned separately. Although discussed in the context of the nebulizer 10 shown in FIGS. 1 and 2, it is contemplated that the present invention may be utilized with other nebulizers and/or devices.

FIG. 3 shows the mesh 28 for the nebulizer 10. As discussed above, mesh 28 has an upper surface 28a (which, when assembled in the upper portion 14, is in fluid communication with mouthpiece 30) and a lower surface 28b (which, when assembled in the upper portion 14, is in fluid communication with the aqueous-based medicament contained in chamber 22). In the exemplary embodiment, the mesh 28 has an outer region 38, an inner region 40, and a central region 42. Outer region 38 has a number of closely spaced apertures 36 and occupies a majority of the surface area of the mesh 28. The inner region 40 has a number of apertures 36 that are more widely spaced as compared with those of the outer region 38. The inner region 40 separates the outer region 38 from the central region 42. As seen in FIG. 3, the central region 42 has no apertures 36 therein.

In the exemplary embodiment, the apertures 36 are tapered having a diameter of approximately 6 μm at the lower surface 28b and having a diameter of approximately 3 μm at the upper surface 28a. It should be noted, however, that the size and amount of taper of the apertures 36 may be varied while remaining within the scope of the present invention.

Nebulizer 10 is often employed to dispense protein based medicaments such as, and without limitation, Alpha-1 Antitrypsin (A1AT), Colistin Sulfate (Colistin), and Recombinant Human Deoxyribonuclease (Dornase Alfa-DNase). As previously discussed, protein based medicaments may accumulate within the nebulizer 10, for example, on the mesh 28. This accumulation may occlude the mesh apertures 36, thereby negatively affecting the performance of the nebulizer 10. Although many known cleaning protocols have been employed in an effort to remove and/or prevent the accumulation of material on or within the nebulizer 10, the results obtained from these know cleaning protocols have been disappointing and unsatisfactory. It has been found, however, that the cleaning protocol of the present invention removes the accumulated material, and furthermore, regular use of the cleaning protocol of the present invention prevents subsequent accumulation.

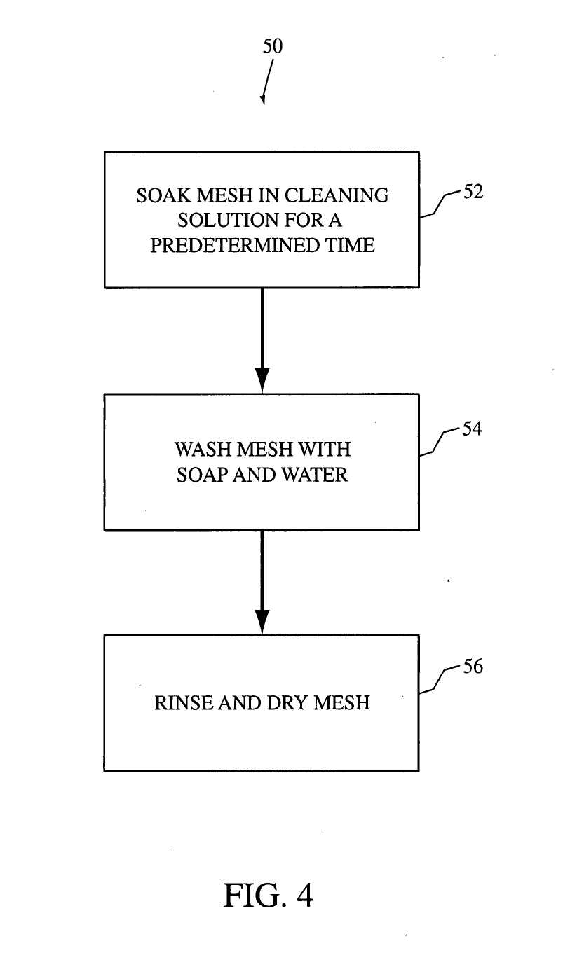

FIG. 4 is a flowchart illustrating an operational process 50 for cleaning the nebulizer (i.e., a cleaning protocol) according to an exemplary embodiment of the present invention. Operational process 50 is discussed in the context of cleaning the nebulizer mesh 28; however, it is contemplated that other components of the nebulizer 10 having an accumulation of material thereon (and/or susceptible to the accumulation of material) may also be cleaned using operational process 50. In the exemplary embodiment, operational process 50 is initiated after the mesh 28 has been removed from the nebulizer 10.

In operation 52, the mesh 28 is soaked, for a predetermined time, in an aqueous cleaning solution having an enzyme dissolved therein. In an exemplary embodiment, for example, the mesh 28 is placed in an aqueous solution having the proteolytic enzyme subtilisin A dissolved in de-ionized water. The solution may also contain, without limitation, buffering and effervescing agents which promote the cleaning process.

Although discussed in the context of employing the proteolytic enzyme subtilisin A and the mixing agent de-ionized water to remove the protein A1AT from the mesh 28, it should be apparent that any suitable enzymes and/or mixing agents may be used, either individually or in combination, to remove a medicament that accumulates on a portion of the nebulizer 10. The cleaning solution may include, for example, proteolytic, carbolytic and lipolytic enzymes. Typical examples include, but are not limited to papain, chymopapain, pancreatin, pepsin, trypsin, chymotrypsin, protease, amylase, lipase, ficin, bromelin, streptokinase, and/or streptodornase. The mixing agent may include, for example and without limitation, tap water, saline solution, distilled water, or a variety of other liquids.

In the exemplary embodiment, the solution is prepared by dissolving a readily available enzymatic cleaning tablet in de-ionized water. More specifically, the solution is prepared by dissolving, in approximately 20 ml of de-ionized water, a single Ultrazyme® Universal enzymatic cleaning tablet manufactured by Advanced Medical Optics.

The predetermined soak time may vary depending on several factors such as, and without limitation, the amount and/or type of protein that has accumulated on the mesh 28, the specific concentration of the enzyme within the aqueous cleaning solution, the specific enzyme that is being used in the aqueous cleaning solution, and/or the rate of reaction between the accumulated material and the specific enzyme being used. Although other predetermined soak times are contemplated, the mesh 28 is soaked in the aqueous cleaning solution for a predetermined time of approximately four (4) hours in the exemplary embodiment. Of course, the amount of time that the mesh is soaked may vary depending on the above noted factors such as the type and/or amount of accumulated protein, type, and/or concentration of the enzyme reaction rate, etc. The present invention contemplates that any suitable soak time could be used without departing from the teachings of the present invention. As such, the soak time may vary considerably from four (4) hours.

After the mesh 28 is removed from the aqueous cleaning solution, operation 54 is performed. In operation 54, the mesh 28 is washed with warm soapy water. The soapy water removes any residual aqueous cleaning solution which, if left on the mesh, may undesirably affect the medicament that comes into contact with the mesh 28 the next time the nebulizer 10 is used by a patient. Although soapy water is used in the current embodiment, another solution and/or agent capable of removing any residual aqueous cleaning solution from the mesh 28 may be used.

After washing the mesh 28 with soap and water, operation 56 is performed where the mesh 28 is rinsed and dried. The rinsing removes any residual soap that may remain from operation 22. In this exemplary embodiment, the mesh 28 is rinsed using tap water and allowed to air dry. However, other rinsing agents and drying methods are contemplated. After the mesh 28 is dry, it may be re-inserted into the nebulizer 10, which may then be reassembled for use.

Although discussed above in the context of separating the mesh 28 from the other parts of the nebulizer 10 (e.g., the chamber lid 26), it is contemplated that the mesh 28 may remain housed within the chamber lid 26 during cleaning. According the combination of the mesh 28 and the chamber lid 26 (and/or any portion thereof) may be cleaned as discussed above in conjunction with FIG. 4.

Gravimetric Output Rate Testing

Observations from a series of tests verified the increased effectiveness of the cleaning protocol of the present invention as compared to a typical cleaning protocol. More specifically, a number of A1AT treatments were completed on each of three nebulizers (referred to herein by their individual serial numbers: FS-216, FS-220, and FS-222). Each A1AT treatment consisted of nebulizing two, 1.3 ml charges of 50 mg/ml A1AT solution.

In the first stage of the test, the mesh from each nebulizer was subjected to a known cleaning protocol (i.e., washed with warm soapy water, rinsed, and dried) after each of several A1AT treatments. The gravimetric output rate of each nebulizer was measured during each A1AT treatment. The gravimetric output rates of the nebulizers whose meshes were subjected to the known cleaning protocol were seen to decrease substantially after a number of A1AT treatments; thereby indicating that the mesh apertures had become occluded due to accumulation of the A1AT material.

In the second stage of the test, five consecutive Salamol treatments (each treatment consisting of 1.3 ml charges of 2.5 mg/ml Salamol solution) were first nebulized in each of the three nebulizers FS-216, FS-220, and FS-222. The Salamol solution is used as a test medicament for comparison purposes. Table 1 shows the Mean, Standard Deviation, and the Coefficient of Variation of Gravimetric Output Rate for each of the three nebulizers FS-216, FS-220, and FS-222 for the five consecutive Salamol treatments.

TABLE 1
Mean, Standard Deviation and Coefficient of Variation
of Gravimetric Output Rate for 1.3 ml Charge of 2.5 mg/ml
Salamol Solution From Five Consecutive Treatments With
Three I-neb Devices Before Testing With A1AT.

| | Device | | | |
|---|---|---|---|---|
| | FS-216 | FS-220 | FS-222 | Overall Mean |
| Mean (g/min) | 0.156 | 0.203 | 0.187 | 0.182 |
| Std. Dev. (g/min) | 0.025 | 0.023 | 0.019 | |
| CV (%) | 16 | 12 | 10 | |

Next, fourteen (14) A1AT treatments were nebulized on each of the three nebulizers (wherein each A1AT treatment consisted of nebulizing two, 1.3 ml charges of 50 mg/ml A1AT solution without cleaning between the two charges). The mesh from each nebulizer was subjected to the cleaning protocol of the present invention between each A1AT treatment. More specifically, after each A1AT treatment, the nebulizer mesh from each of the three nebulizers was soaked for a predetermined time (e.g., approximately four (4) hours) in a cleaning solution prepared by dissolving a commercially available, enzymatic cleaning tablet (e.g., an Ultrazyme® Universal enzymatic cleaning tablet) in a mixing agent (e.g., de-ionized water). After soaking for the predetermined time, the meshes were washed in warm water, rinsed with tap water, and air dried.

In the test, the gravimetric output rate of each nebulizer was measured during each of the fourteen (14) A1AT treatments. Table 2 shows the Mean, Standard Deviation, and the Coefficient of Variation of Gravimetric Output Rate for each of the three nebulizers FS-216, FS-220, and FS-222 for the A1AT treatments.

TABLE 2
Mean, Standard Deviation and Coefficient of Variation
of Gravimetric Output Rate of 50 mg/ml A1AT, 1.3 ml
Charge Run Twice With Cleaning Between From Fourteen
Consecutive Treatments With Three I-neb Devices

| | Device | | | |
|---|---|---|---|---|
| | FS-216 | FS-220 | FS-222 | Overall Mean |
| Mean (g/min) | 0.158 | 0.187 | 0.181 | 0.175 |
| Std. Dev. (g/min) | 0.030 | 0.026 | 0.031 | |
| CV (%) | 19 | 14 | 17 | |

As seen in Table 2, the gravimetric output rates of the nebulizers whose meshes were subjected to the improved cleaning protocol of the present invention remained substantially constant after the fourteen treatments; thereby indicating that the mesh apertures were not occluded due to accumulation of A1AT material.

After the fourteen (14) A1AT treatments were concluded, five more consecutive Salamol treatments (each treatment consisting of 1.3 ml charges of 2.5 mg/ml Salamol solution) were nebulized in each of the three nebulizers FS-216, FS-220, and FS-222. Table 3 shows the Mean, Standard Deviation, and the Coefficient of Variation of Gravimetric Output Rate for each of the three nebulizers FS-216, FS-220, and FS-222 for the five consecutive Salamol treatments conducted after the fourteen (14) A1AT treatments.

TABLE 3
Mean, Standard Deviation and Coefficient of Variation
of Gravimetric Output Rate of 2.5 mg/ml Salamol Solution
From Five Consecutive Treatments With Three I-neb
Devices After Testing With A1AT.

| | Device | | | |
|---|---|---|---|---|
| | FS-216 | FS-220 | FS-222 | Overall Mean |
| Mean (g/min) | 0.260 | 0.264 | 0.186 | 0.237 |
| Std. Dev. (g/min) | 0.045 | 0.059 | 0.038 | |
| CV (%) | 17 | 22 | 21 | |

Comparing the results in Table 3 to those in Table 1, it is surprising to note that the gravimetric output rate of the meshes actually increased due to the cleaning protocol of the present invention. More specifically, comparing the overall mean from Table 3 to the overall mean from Table 1, it can be seen that the overall mean of the gravimetric output rate of the Salamol solutions surprisingly increased by approximately 30% as a result of the cleaning protocol of the present invention.

High Resolution Images

In addition to measuring the gravimetric output rates of the nebulizers, high resolution images were taken of several meshes subjected to various cleaning protocols and after various stages of treatment. The high resolution images corroborated the results of the gravimetric output rate tests. More specifically, high resolution images of meshes which were not subjected to a cleaning protocol and/or which were subjected to a known cleaning protocol showed accumulation occluding the mesh apertures (i.e., thereby corroborating the reduction in the gravimetric output rates). Furthermore, high resolution images of meshes which were subjected to the cleaning protocol of the present invention showed little or no accumulation on the mesh apertures (i.e., thereby corroborating the sustained and/or increased gravimetric output rates).

No A1AT Treatment, No Cleaning

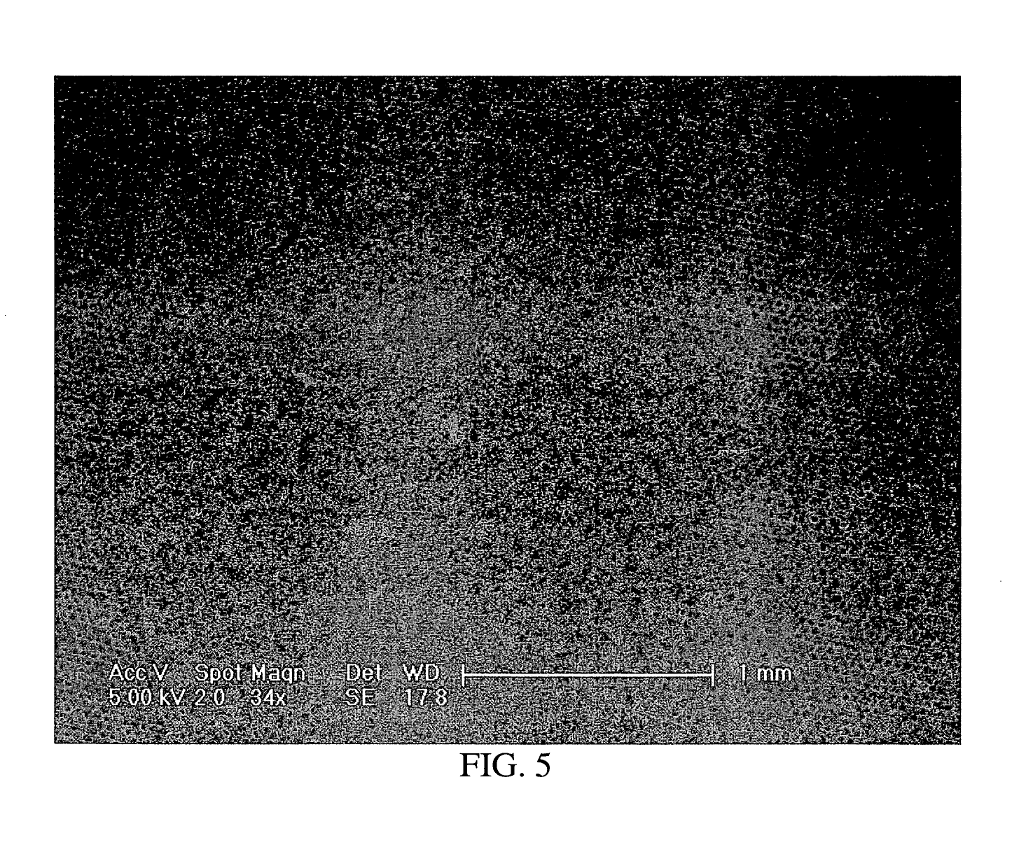

Prior to running any treatments, a high resolution (i.e., 34×) image of the upper surface of a single mesh was taken. Referring to FIG. 5, it may be observed that the upper surface of the mesh is free of any accumulation and the apertures are not occluded.

Single A1AT Treatment, No Cleaning

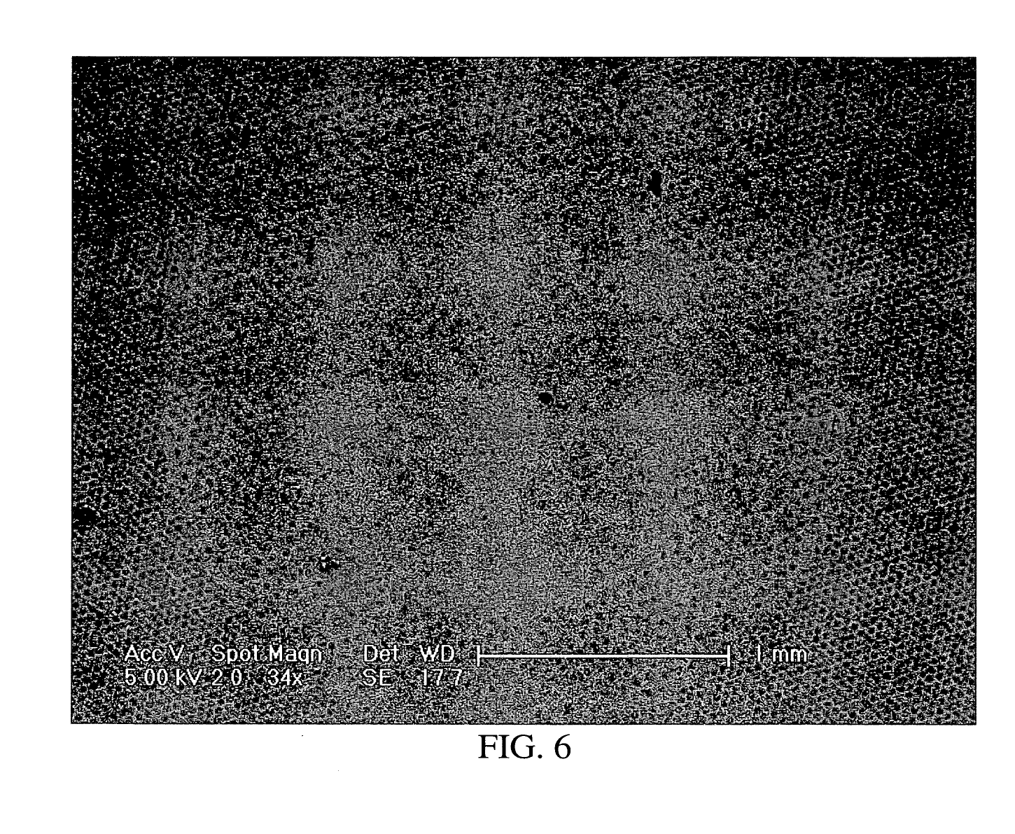

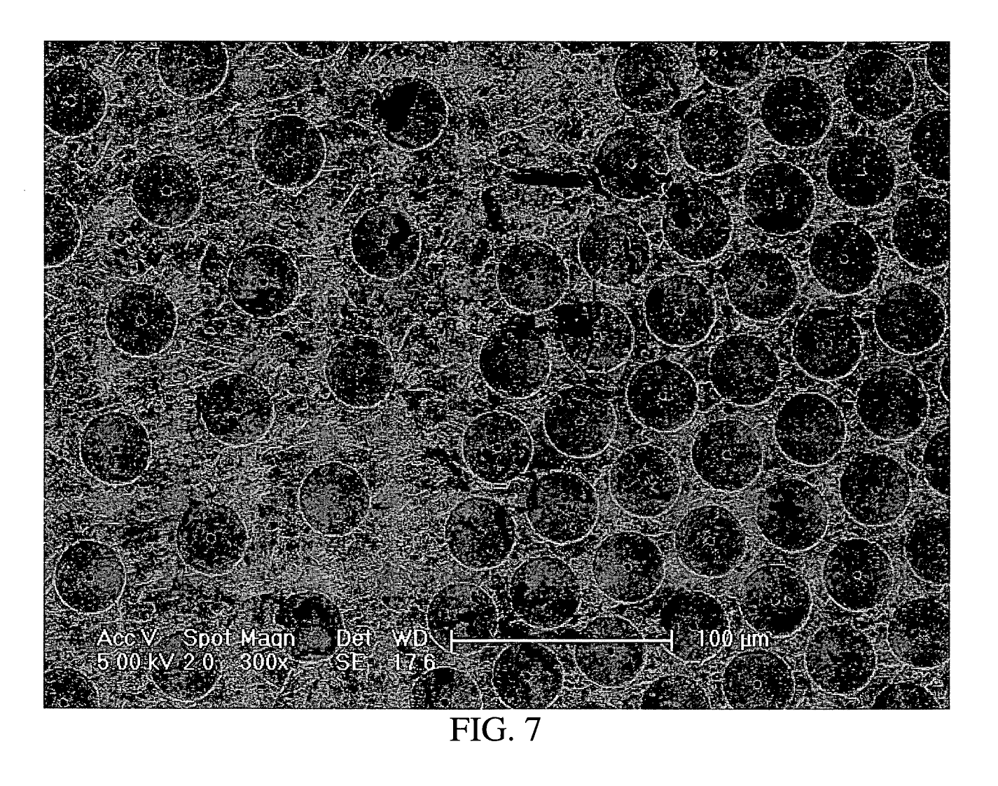

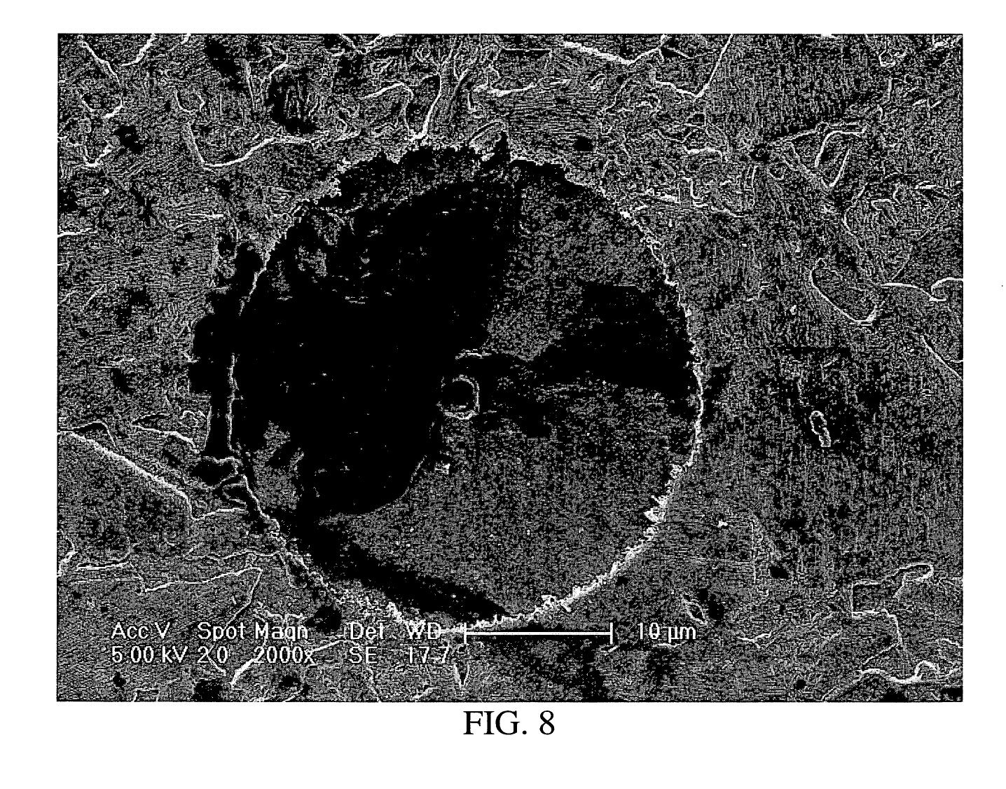

A single treatment of the A1AT solution was nebulized and another high resolution (i.e., 34×) image was taken of the upper surface of mesh. Referring to FIG. 6, it may be observed that the upper surface of the mesh has a small amount of material accumulated mainly near the interface between the inner region and the outer region. A higher magnification (i.e., 300×) image (see FIG. 7) of this region shows that a few apertures are partially occluded by accumulated material. A very high magnification (i.e., 2000×) image (see FIG. 8) illustrates that this accumulation is only a thin layer as some of the mesh surface features remain visible.

Ten A1AT Treatments, No Cleaning

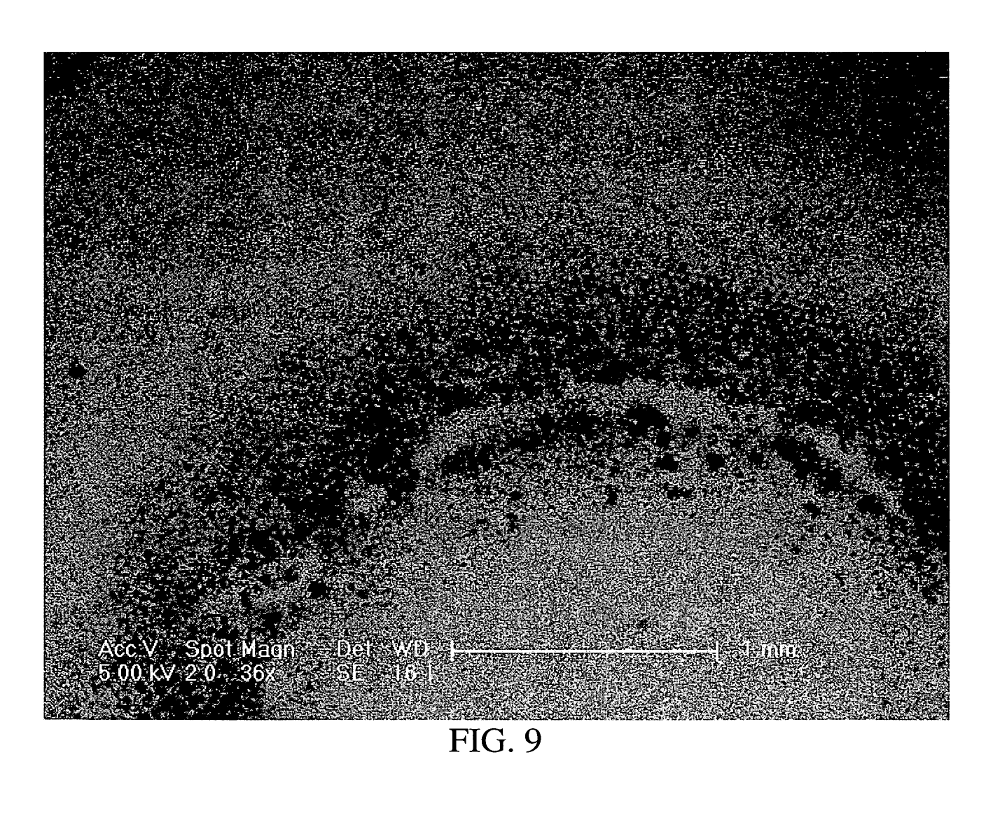

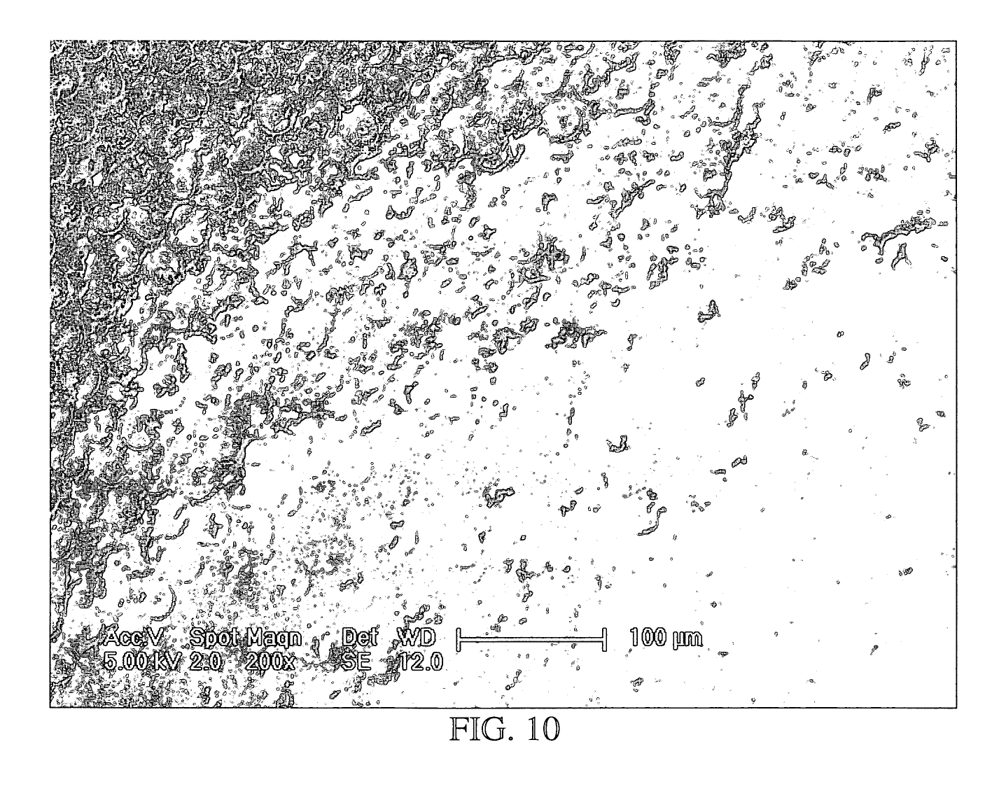

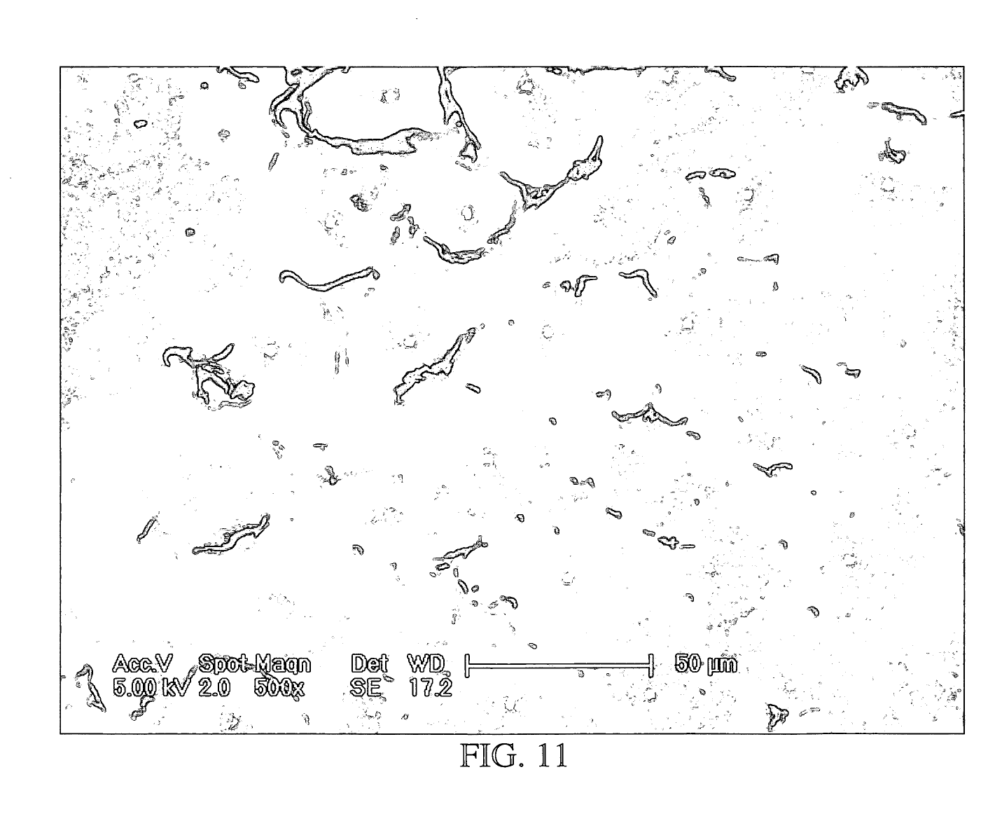

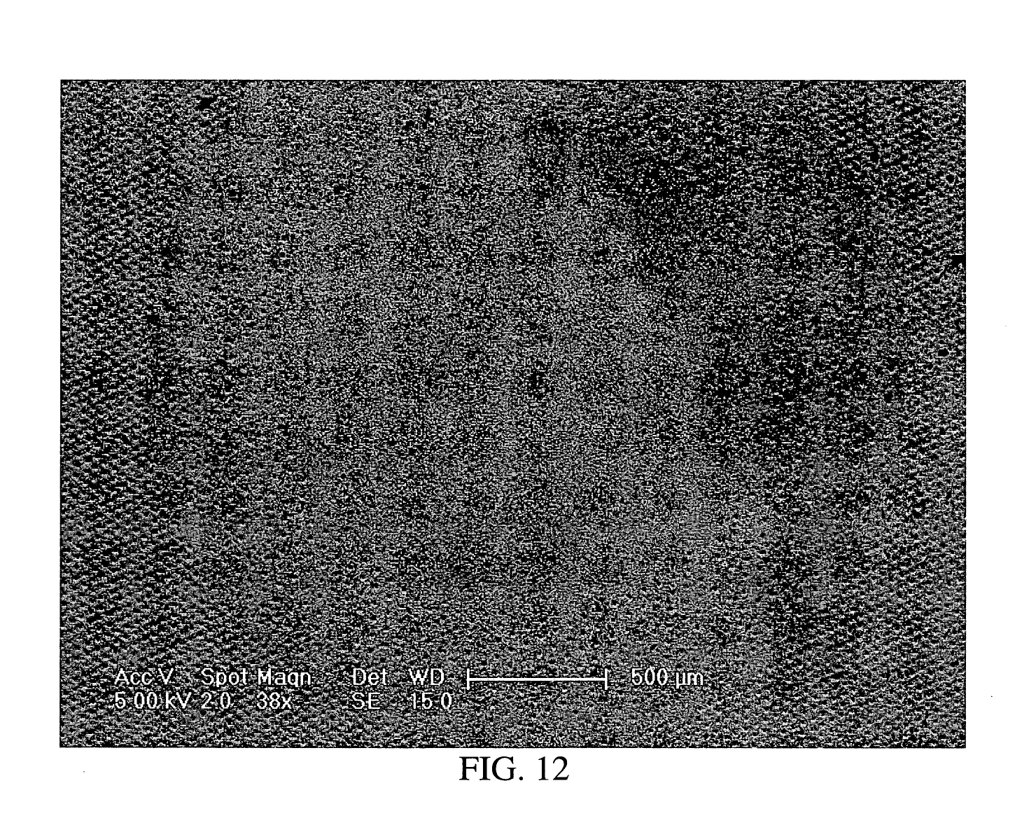

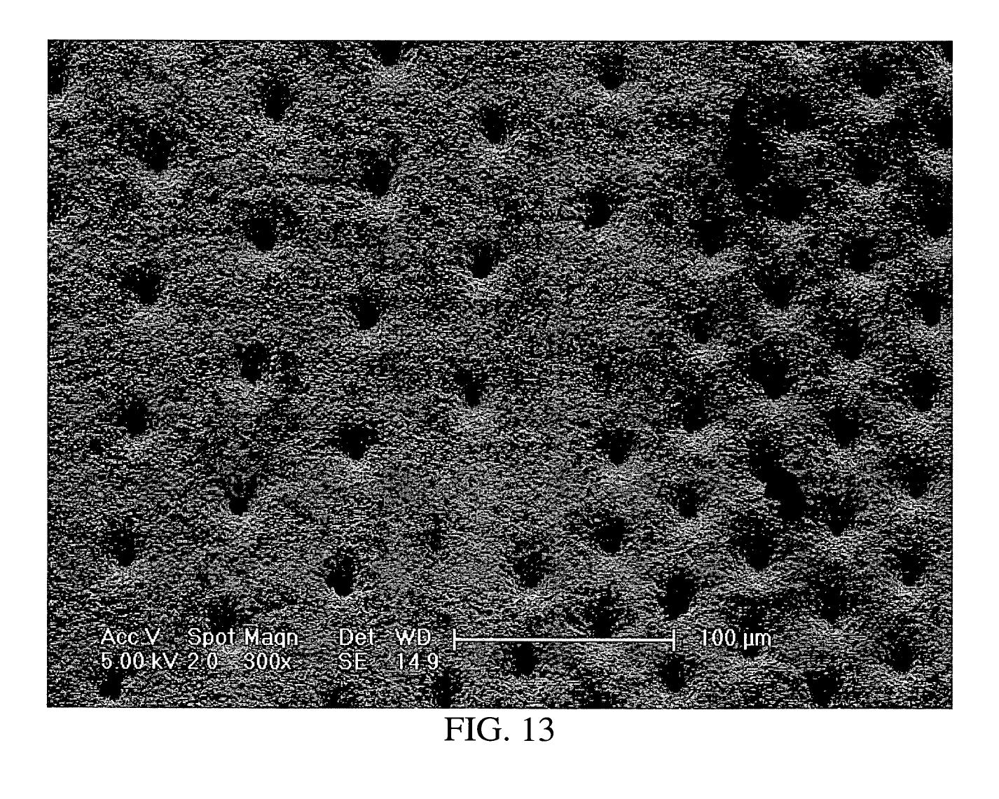

After ten (10) treatments of the A1AT solution were nebulized, without cleaning between each treatment, a high resolution (i.e., 36×) image was taken of the upper surface of the mesh. Referring to FIG. 9, it may be observed that a large amount of material has accumulated over the inner region and the inner half of the outer region. A high magnification (i.e., 200×) image (see FIG. 10) of this area shows that the accumulation is a very thick layer. A higher magnification (i.e., 500×) image (see FIG. 11) of this same area shows that many apertures are completely occluded by accumulated material. High resolution (i.e., 38× and 300×) images of the bottom surface of the mesh were also taken. Referring to FIGS. 12 and 13, it may be observed that accumulation of the A1AT is generally limited to the upper surface of the mesh.

Ten A1AT Treatments, Soapy Water Cleaning

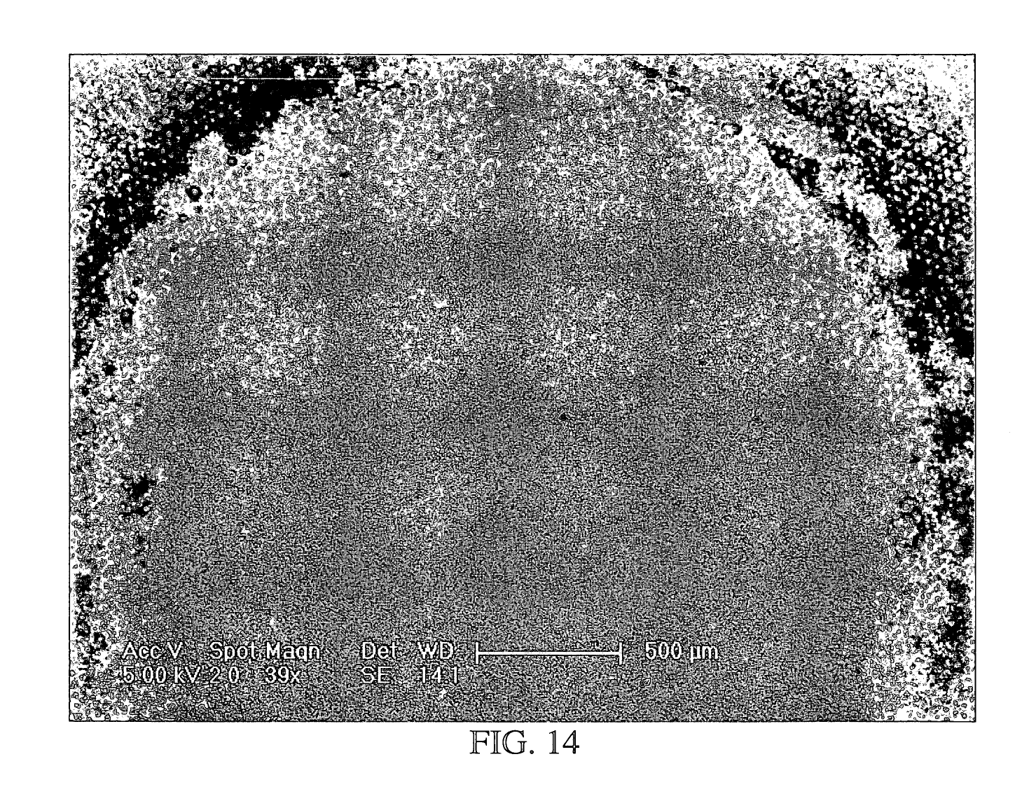

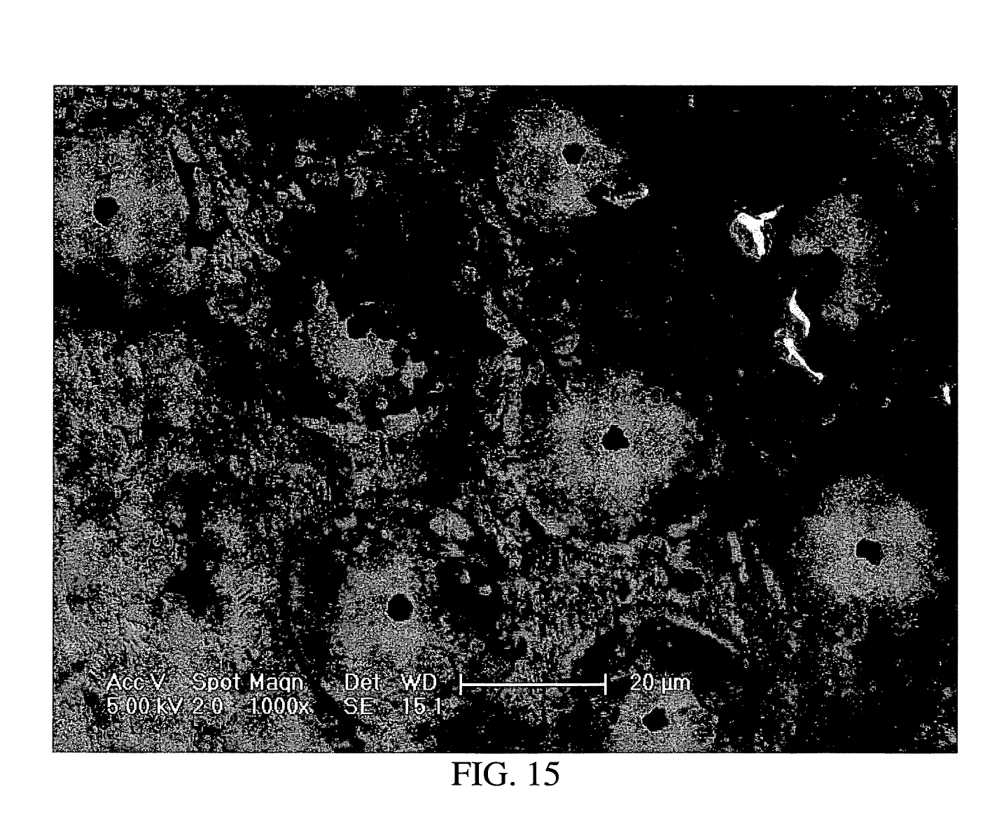

Ten A1AT treatments were nebulized with the mesh being washed in warm soapy water, rinsed, and air dried according to a known cleaning protocol between each treatment. After the final air drying, a high resolution (i.e., 39×) image was taken of the upper surface of mesh. Referring to FIG. 14, it may be observed that a large amount of material remains accumulated over the inner region and the inner half of the outer region. A higher magnification (i.e., 1000×) image (see FIG. 15) of this area shows that many apertures remain completely occluded by the accumulated material. Additionally, the accumulation is observed to be thick enough to obscure the underlying relief of the mesh. The images illustrated in FIGS. 14 and 15 correlate with the results obtained during the first stage of the gravimetric tests discussed above (i.e., the gravimetric output rates of the nebulizers whose meshes were subjected to the known cleaning protocol were seen to decrease substantially due to accumulation of the A1AT material).

Ten A1AT Treatments, Four Hour Protease Soak Cleaning

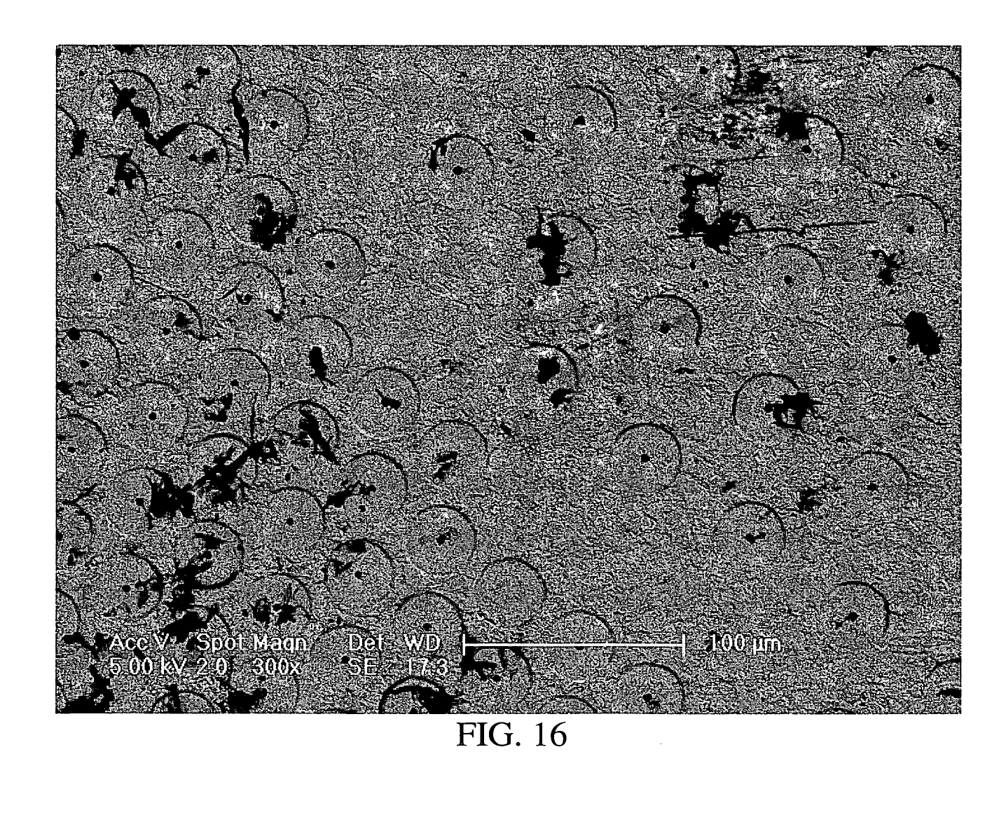

A mesh, which was subjected to ten (10) treatments of the A1AT solution, was cleaned by soaking in a protease solution for a predetermined time of approximately four (4) hours. After soaking, a high resolution (i.e., 300×) image was taken of a portion of the upper surface of mesh. Referring to FIG. 16, it may be observed that almost all of the accumulated material has been removed from the mesh. Surprisingly, the removal is so complete that few, if any, of the apertures 36 remained completely occluded by the accumulated material. Additionally, the underlying relief of the mesh may be observed thereby indicating that the remaining accumulation is present only as a thin layer. FIG. 16 illustrates that the use of a protease solution cleaning protocol in accordance with the present invention is far superior to not cleaning the mesh (the result of which are shown in FIGS. 9-11) and/or to using the prior art cleaning protocol (the result of which are shown in FIGS. 14 and 15).

Fifteen Double Run A1AT Treatments, Four Hour Protease Soak Cleaning Between Each Double Run

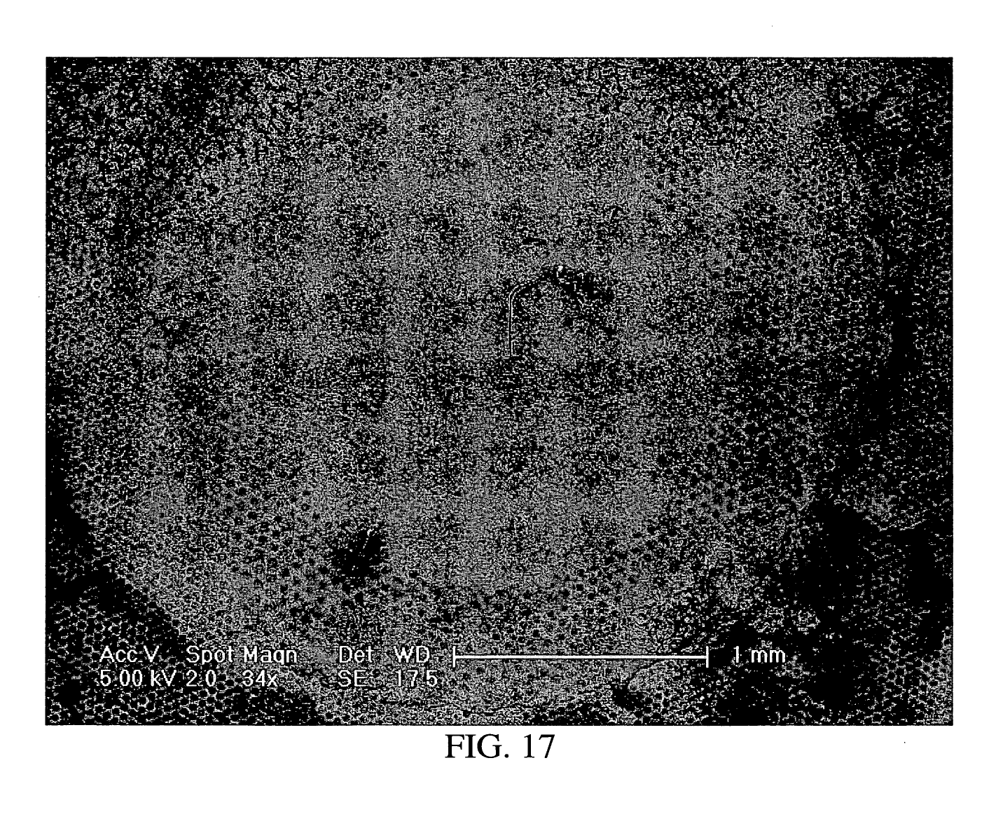

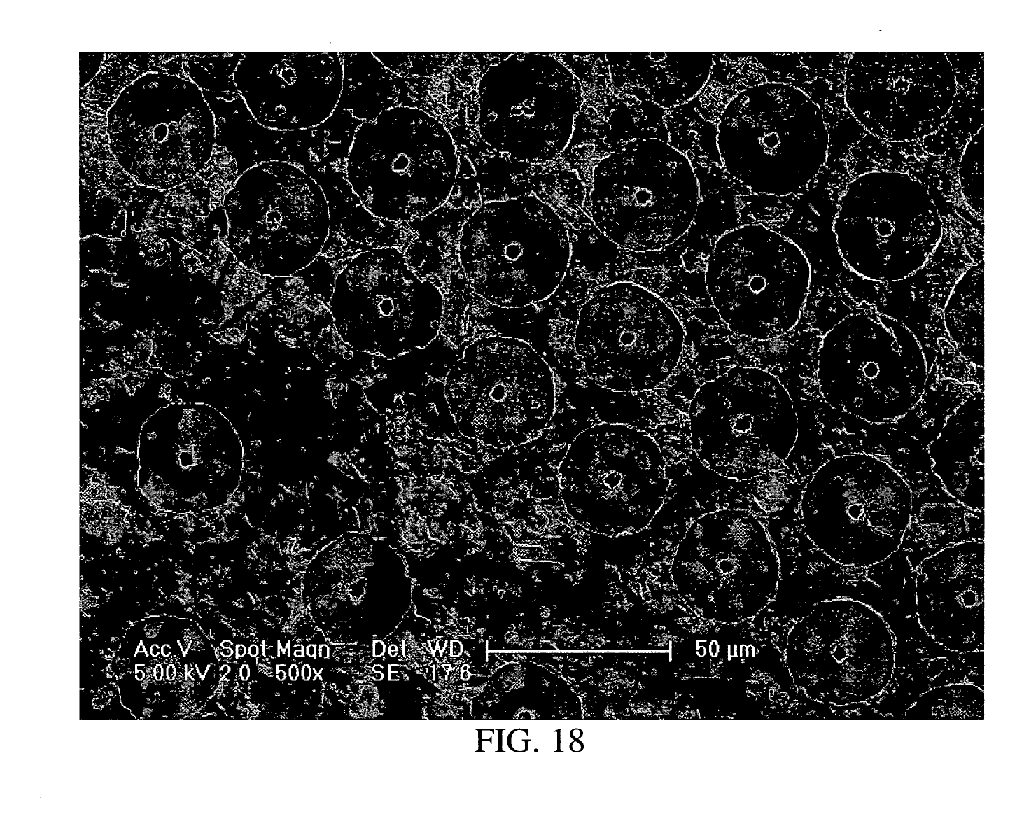

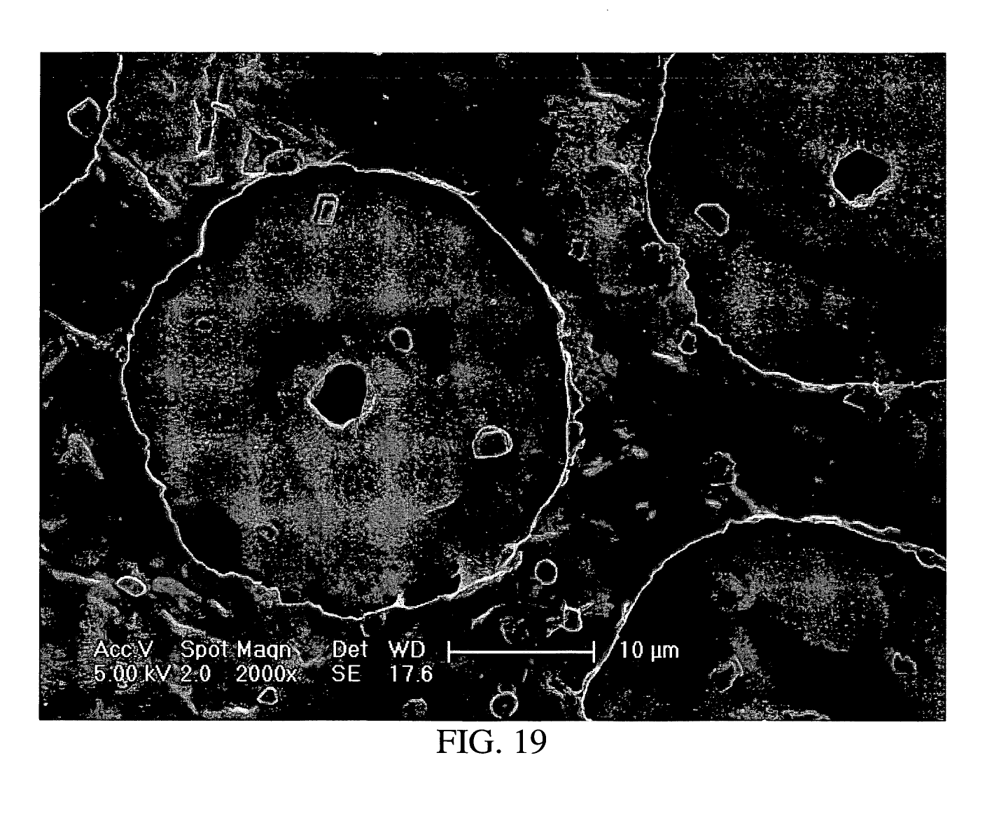

A mesh, which was subjected to fifteen (15) double treatments of the A1AT solution, was soaked in a protease solution for a predetermined time of approximately four (4) hours between each double run. More specifically, two runs of 1.3 ml of 50 mg/ml A1AT solution were nebulized. The mesh was then removed from the nebulizer and soaked in a protease solution for approximately four (4) hours. These steps were repeated fifteen (15) times. After subjecting the mesh to the fifteenth, four (4) hour soak, a high resolution (i.e., 34×) image was taken of the upper surface of mesh. Referring to FIG. 17, it may be observed that a minor amount of accumulated material remains dispersed over the upper surface of the mesh. Higher magnification (i.e., 500× and 2000×) images (see FIGS. 18 and 19) of these regions, however, reveals that the remaining accumulation is very thin. For example, any observed accumulation is not thick enough to obscure the underlying relief of the mesh. Significantly, none of the apertures are occluded by the accumulated material. FIG. 17 also illustrates that the use of a protease solution cleaning protocol in accordance with the present invention is far superior to not cleaning the mesh (see FIGS. 9-11) and/or to using the prior art cleaning protocol (see FIGS. 14 and 15).

### CONCLUSION

The images discussed above reveal that the top surface of a mesh shows some signs of contamination and a few of the apertures may be partially occluded following a single treatment with A1AT. This correlates with previous experiments where the gravimetric output rate remains relatively high after a small number of A1AT treatments.

The images also reveal that the accumulation of material becomes quite extensive and, in some places, quite thick after a number of treatments with A1AT (e.g., approximately ten (10) treatments). In fact, many apertures were completely occluded. This too correlates with the significant drop in gravimetric output rate as seen in previous experiments.

Furthermore, the images reveal that once heavily contaminated with A1AT, the use of the known cleaning protocol of washing the mesh in warm soapy water followed by rinsing with tap water and drying in air had no appreciable effect (i.e., the accumulation of A1AT remained). However, a cleaning protocol according to an exemplary embodiment of the present invention exhibited markedly improved results: a majority of the accumulated material was removed from the mesh. More specifically, soaking the mesh in a protease solution for a predetermined time removed a majority of the accumulated material and reduced the thickness of any material that happened to remain. After employing the cleaning protocol of the present invention, surprisingly few, if any, of the apertures remained occluded, even partially.

In addition to removing material that has already accumulated on the mesh, employing the cleaning protocol according to the exemplary embodiment between A1AT treatments prevents material from accumulating on the mesh. More specifically, the images discussed above show that soaking the mesh in a protease solution for a predetermined time between treatments limited accumulation of A1AT material to small, localized areas. Additionally any material that did accumulate was thin and very few apertures, if any, were occluded (those that were tended to be only partially occluded). These results correlate with the observation that the gravimetric output rate remained high for meshes subjected to the cleaning protocol of the present invention.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. Accordingly, the particular arrangements disclosed are meant to be illustrative only and not limiting as to the scope of the invention which is to be given the full breadth of the claims appended and any and all equivalents thereof.

What is claimed is:

1. A method for cleaning a medicament from at least a portion of a nebulizer comprising:
soaking the portion of the nebulizer for a predetermined time in a cleaning solution having an enzyme dissolved therein;
washing the cleaning solution from the portion of the nebulizer; and
rinsing the portion of the nebulizer after said washing.

2. The method of claim 1 wherein the enzyme dissolved therein is selected from the group comprising subtilisin, subtilisin A, papain, chymopapain, pancreatin, pepsin, trypsin, chymotrypsin, protease, amylase, lipase, ficin, bromelin, streptokinase, or streptodomase.

3. The method of claim 1 further comprising selecting the predetermined time in response to at least one of an amount of the medicament accumulated on the portion of the nebulizer, a type of the medicament accumulated on the portion of the nebulizer, a concentration of the enzyme dissolved within the cleaning solution, a type of enzyme dissolved within the cleaning solution, and a rate of reaction between the medicament accumulated on the portion of the nebulizer and a type of enzyme dissolved within the cleaning solution.

4. The method of claim 1 wherein the medicament is Alpha-1 Antitrypsin and wherein the method further comprises soaking the portion of the nebulizer in a cleaning solution having the enzyme subtilisin A dissolved therein for approximately four hours.

5. The method of claim 1 wherein the portion of the nebulizer includes a mesh and wherein the method further comprises separating the mesh from the nebulizer.

6. The method of claim 1 further comprising drying the portion of the nebulizer after the step of rinsing.

7. The method of claim 1 wherein the step of soaking the portion of the nebulizer in a cleaning solution for a predetermined time further comprises preparing the cleaning solution.

8. The method of claim 7 wherein the step of preparing the cleaning solution further comprises dissolving at least one of a proteolytic enzyme, a carbolytic enzyme, or an lipolytic enzyme in a mixing agent.

9. The method of claim 7 wherein the preparing the cleaning solution comprises dissolving an enzymatic cleaning tablet in a mixing agent.

10. A method for removing a protein from a nebulizer mesh comprising:
soaking the mesh in a cleaning solution for a predetermined time, the cleaning solution having a proteolytic enzyme dissolved therein;
washing the cleaning solution from the mesh; and
rinsing the mesh after said washing.

11. The method of claim 10 wherein the proteolytic enzyme dissolved therein is selected from the group comprising subtilisin, subtilisin A, papain, chymopapain, pepsin, protease, of bromelin.

12. The method of claim 10 further comprising selecting the predetermined time based on at least one of an amount of the protein that has accumulated on the mesh, a type of the protein that has accumulated on the mesh, a concentration of the enzyme dissolved within the cleaning solution, a type of enzyme that is dissolved within the cleaning solution, and a rate of reaction between the protein accumulated on the portion of the nebulizer and a type of enzyme dissolved within the cleaning solution.

13. The method of claim 10 wherein the protein is Alpha-1 Antitrypsin and wherein the method further comprises soaking the mesh in a cleaning solution having the enzyme subtilisin A dissolved therein for approximately four hours.

14. The method of claim 10 further comprising drying the mesh after the rinsing step.

15. The method of claim 10 wherein the washing step includes washing the cleaning solution from the mesh with a solution of soap and water.

16. The method of claim 10 wherein the step of soaking the mesh in a cleaning solution for a predetermined time further comprises preparing the cleaning solution.

17. The method of claim 16 wherein the step of preparing the cleaning solution comprises dissolving an enzymatic cleaning tablet in a mixing agent.
